(19) United States
(12) Patent Application Publication
(10) Pub. No.: US 2018/0110461 A1
(43) Pub. Date: Apr. 26, 2018

(54) AUDIO SIGNAL PROCESSING DEVICE, AUDIO SIGNAL PROCESSING METHOD, AND STORAGE MEDIUM

(71) Applicant: YAMAHA CORPORATION, Hamamatsu-shi (JP)

(72) Inventors: Kiyoshi YAMAKI, Iwata-shi (JP); Morito MORISHIMA, Fukuroi-shi (JP); Atsushi ISHIHARA, Iwata-shi (JP); Takehiko KAWAHARA, Hamamatsu-shi (JP)

(21) Appl. No.: 15/850,649

(22) Filed: Dec. 21, 2017

Related U.S. Application Data
(63) Continuation of application No. PCT/JP2016/067980, filed on Jun. 16, 2016.

(30) Foreign Application Priority Data
Jun. 29, 2015 (JP) 2015-130156

(51) Int. Cl. A61B 5/00; A61B 5/08; A61B 5/0482; A61B 5/024

(52) U.S. Cl. CPC A61B 5/4809; A61B 5/0816; A61B 5/024; A61B 5/0482; A61B 5/4812

(57) ABSTRACT

An audio signal processing device (20) includes: an acquirer (210) configured to acquire biological information of a human subject; a processor (240) configured to identify at least one of a breathing cycle and a heartbeat cycle of the human subject, based on the biological information; and an effect imparter (250) configured to impart to an audio signal (SD) a frequency characteristic that changes at a cycle set in accordance with either the breathing cycle or the heartbeat cycle.

### TECHNICAL FIELD

The present invention relates to an audio signal processing device, an audio signal processing method, and a storage medium.

### BACKGROUND ART

Recently, there have been proposed technologies for enhancing sleep or imparting a relaxing effect by detecting biological information of body motion, breathing, heartbeat, etc., and generating a sound in accordance with the detected biological information (for example, refer to Japanese Patent Application Laid-Open Publication No. H4-269972). Moreover, there have also been proposed technologies for adjusting, in accordance with a relaxation state of a human subject, at least one of a type, a volume, and a tempo of a generated sound (for example, refer to Japanese Patent Application Laid-Open Publication No. 2004-344284).

When a sound is generated to enhance sleep of a person hearing the sound (hereinafter, “human subject”), if a monotonous sound is generated, the sound for improving sleep may impede or disturb the sleep due to reasons such as the human subject becoming bored by the sound or the sound becoming annoying to the human subject.

### SUMMARY OF INVENTION

The present invention has been made in consideration of the aforementioned circumstances, and one of the problems to be solved by the present invention is to provide a technology by which occurrences of sleep disturbance caused by sound are minimized. Such occurrences of sleep disturbance are contrary to the expected effect of improving sleep by use of sound.

To solve the aforementioned problem, in one aspect, an audio signal processing device of the present invention includes: an acquirer configured to acquire biological information of a human subject; a processor configured to identify at least one of a breathing cycle and a heartbeat cycle of the human subject, based on the biological information; and an effect imparter configured to impart to an audio signal a frequency characteristic that changes at a cycle that is in accordance with either the breathing cycle or the heartbeat cycle.

In another aspect, an audio signal processing device according to the present invention includes: an acquirer configured to acquire biological information of a human subject; an estimator configured to estimate a sleep state based on the biological information; a processor configured to decide a vibrato cycle in accordance with the sleep state estimated by the estimator; and an effect imparter configured to impart to an audio signal a vibrato effect corresponding to the vibrato cycle decided by the processor.

### BRIEF DESCRIPTION OF THE DRAWINGS

FIG. 1 is a diagram showing an overall configuration of a system including an audio signal processing device according to a first embodiment.

FIG. 2 is a block diagram showing a functional configuration of the audio signal processing device according to the first embodiment.

FIG. 3 is a block diagram showing an example configuration of an audio signal generator.

FIG. 4 is an explanatory diagram showing an example of time changes in a cutoff frequency of a low-pass filter.

FIG. 5 is a timing chart showing a relationship among a waveform of sound data, time changes in a cutoff frequency of the low-pass filter, and trigger signals.

FIG. 6 is a flowchart showing a flow of an operation of the audio signal processing device according to the first embodiment.

FIG. 7 is a block diagram showing a functional configuration of an audio signal processing device according to a second embodiment.

FIG. 8 is a flowchart showing a flow of an operation of the audio signal processing device according to the second embodiment.

FIG. 9 is an explanatory diagram that explains control patterns of a frequency characteristic in an audio signal processing device according to one modification.

### MODES FOR CARRYING OUT THE INVENTION

In the following, embodiments of the present invention will be explained with reference to the drawings.

### First Embodiment

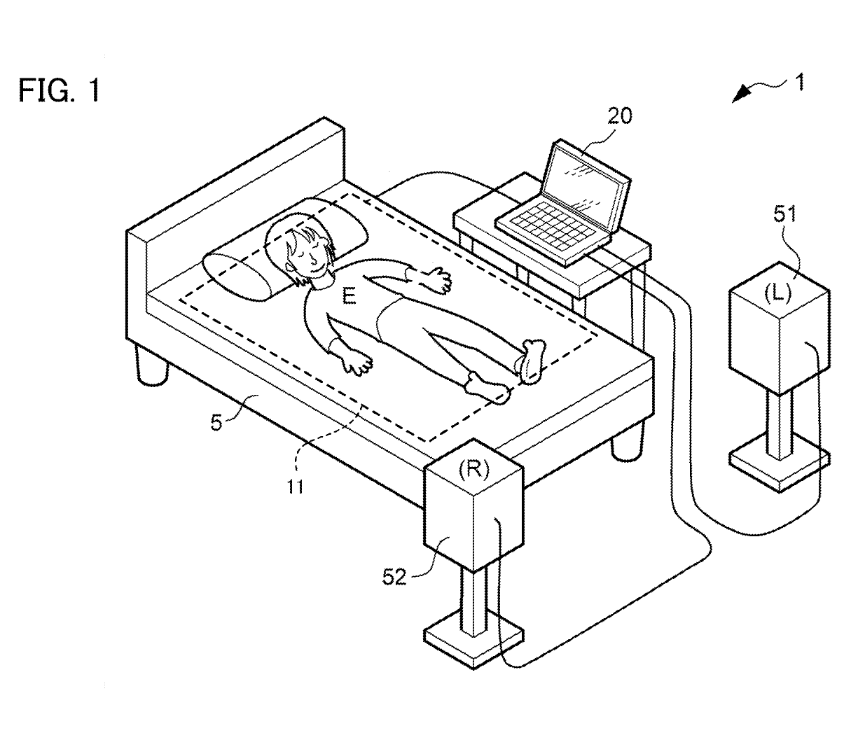

FIG. 1 is a diagram showing an overall configuration of a system 1 including an audio signal processing device 20 according to a first embodiment. As shown in FIG. 1, the system 1 is configured to include a sensor 11, the audio signal processing device 20, and speakers 51 and 52. The system 1 aims to enhance, for example, sleep of a human subject E by causing a sound, which is output by the speakers 51 and 52, to be heard by the human subject E lying on his/her back on a bed 5.

The sensor 11 may be, for instance, a sheet-like piezoelectric element. The sensor 11 is disposed, for example, underneath a mattress on the bed 5. When the human subject E lies down on the bed 5, the sensor 11 detects biological information of the human subject E. The sensor 11 detects body motion resulting from biological activities, including breathing and a heartbeat, of the human subject E. The sensor 11 outputs a detected signal on which components of such biological activities are superimposed. For the sake of convenience, FIG. 1 shows a configuration in which the detected signal is transmitted to the audio signal processing device 20 via a wired connection; however, a configuration in which the detected signal is transmitted wirelessly may be employed instead.

The audio signal processing device 20 is capable of acquiring a breathing cycle BRm, a heartbeat cycle HRm, and body motion of the human subject E based on a detected signal (biological information) output from the sensor 11. The audio signal processing device 20 is, for example, a portable terminal, a personal computer, or the like.

The speakers 51 and 52 are arranged at positions such that the human subject E lying on his/her back perceives sound in stereo. The speaker 51 amplifies a left (L) audio signal of stereo signals output from the audio signal processing device 20 by means of a built-in amplifier, and outputs a sound corresponding to the amplified L audio signal of the stereo signals. Likewise, the speaker 52 amplifies a right (R) audio signal of stereo signals output from the audio signal processing device 20 by means of a built-in amplifier, and outputs a sound corresponding to the amplified R audio signal of the stereo signals. Alternatively, headphones may be employed to provide the human subject E with the sound. However, in the present embodiment, a configuration in which the speakers 51 and 52 are used is employed.

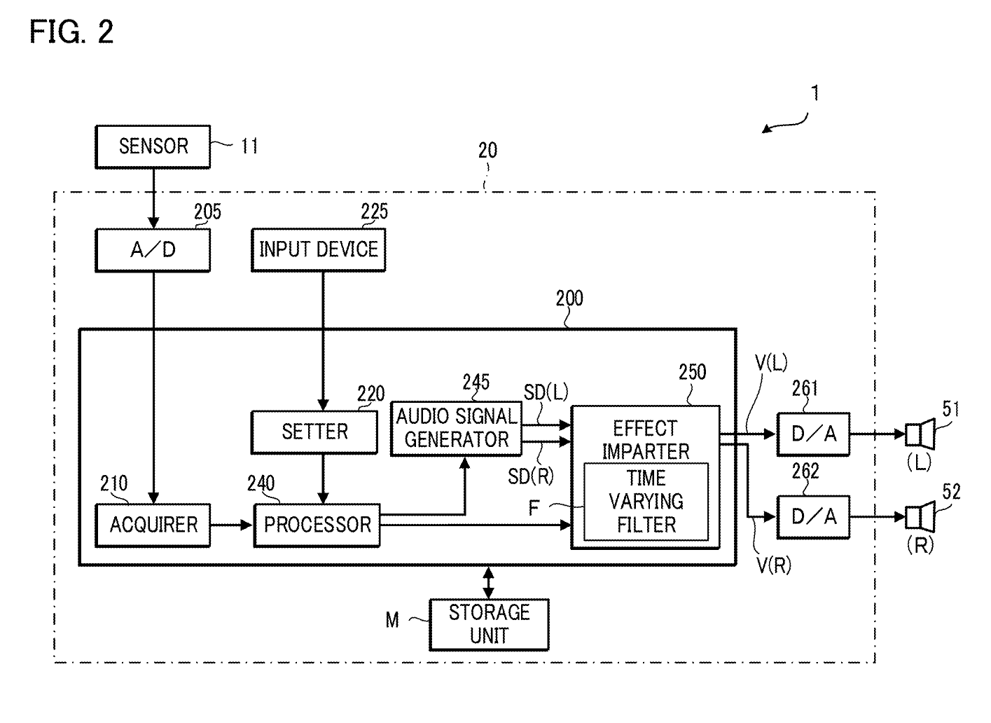

FIG. 2 is a diagram mainly showing a configuration of functional blocks in the audio signal processing device 20, in the system 1. As shown in FIG. 2, the audio signal processing device 20 includes an A/D converter 205, a controller 200, a storage unit M, an input device 225, and D/A converters 261 and 262.

The storage unit M is a non-transitory storage medium, for example, and may be an optical storage medium, such as a CD-ROM (optical disc), or well-known storage media, such as a magnetic storage medium or a semiconductor storage medium. In the present description, a “non-transitory” storage medium encompasses all types of computer-readable storage media with the exception of a transitory, propagating signal, and does not exclude volatile storage media. A program to be executed by the controller 200 and a variety of data for use by the controller 200 are stored in the storage unit M. For example, a plurality of sound information pieces are stored in the storage unit M. The sound information may also be referred to as “sound content”. The sound information (sound content) is, for example, sound information (sound content) related to sound generation. The program may be provided via a communication network (not shown), and then installed in the storage unit M.

The input device 225 is an input/output device in which a display unit (e.g., a liquid-crystal display panel) and an input unit are formed integrally. The display unit displays various images under control of the controller 200, and the input unit is used by a user (e.g., the human subject) to input instructions to the audio signal processing device 20. A touch panel is an example of the input device 225. A device having a plurality of operation elements provided separately from a display unit may be employed as the input device 225.

The controller 200 is constituted of a processing device (such as a CPU), for example. The controller 200, by executing the program stored in the storage unit M, functions as an acquirer 210, a setter 220, a processor 240, an audio signal generator 245, and an effect imparter 250. The entirety or some of these functions may be realized by dedicated electronic circuitry. For example, the audio signal generator 245 and the effect imparter 250 may be configured by large scale integration (LSI). The audio signal generator 245 generates audio signals SD (SD(L) and SD(R)) from the sound information stored in the storage unit M. Each sound information (sound content) stored in the storage unit M may be any sound information in so far as the audio signal generator 245 is able to generate therefrom the audio signal SD. The sound information may be one piece of sound information only. Performance data representing performance information (e.g., notes and pitches), parameter data representing parameters and the like used to control the audio signal generator 245, and waveform data of sound are examples of the sound information. More specific examples of the sound information are: sound information representing a sound of ocean waves (e.g., waveform data representing a sound of ocean waves); sound information representing a bell sound (e.g., waveform data representing a bell sound); sound information representing a guitar sound (e.g., waveform data representing a guitar sound); and sound information representing a piano sound (e.g., waveform data representing a piano sound).

The A/D converter 205 converts the detected signals output by the sensor 11 to digital signals. The acquirer 210 temporarily accumulates the digital detected signals in the storage unit M, for example. The setter 220 is used to carry out a variety of settings. The audio signal processing device 20 generates various types of audio signals V so that the human subject E does not become bored by the sound. The audio signal processing device 20 is capable of outputting sounds from the speakers 51 and 52, the sounds corresponding to the audio signals V.

The setter 220 can select and set a sound information piece to be played (output), from among a large number of sound information pieces stored in the storage unit M, depending on an input operation performed by the human subject E on the input device 225. Specifically, the setter 220 receives from the input device 225 operation information that is in accordance with the input operation performed by the human subject E on the input device 225. The setter 220 supplies to the processor 240 setting data indicative of sound information to be played in accordance with the operation information.

The processor 240 supplies to the audio signal generator 245 a sound information instruction instructing sound information to be played, in accordance with the setting data received from the setter 220.

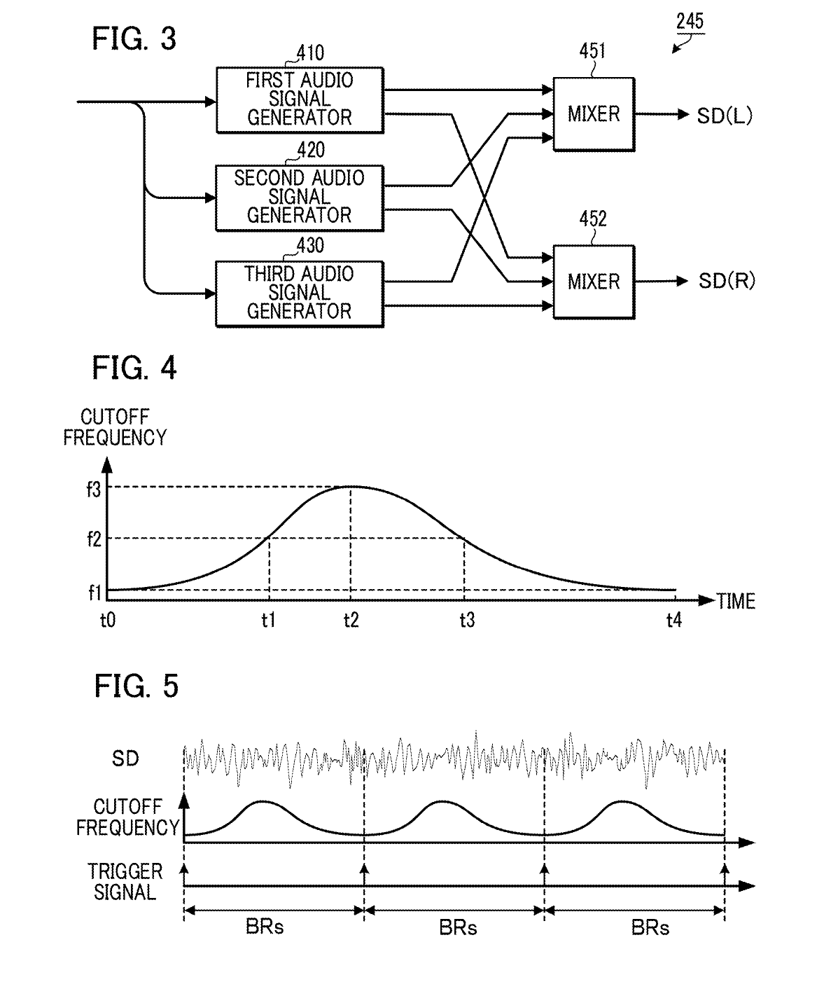

The audio signal generator 245 acquires from the storage unit M sound information corresponding to the sound information instruction. The audio signal generator 245 generates the audio signal SD based on the acquired sound information. The audio signal SD may also be referred to as “sound content”. FIG. 3 shows a detailed configuration of the audio signal generator 245. The audio signal generator 245 includes a first audio signal generator 410, a second audio signal generator 420, a third audio signal generator 430, and mixers 451 and 452. In the present example, the audio signal processing device is capable of concurrently outputting three kinds of sounds. For example, when the input device 225 receives an input operation designating three kinds of sound information pieces, the input device 225 supplies to the setter 220 operation information corresponding to the input operation.

Upon receipt at the setter 220 of the operation information, the setter 220 supplies to the processor 240 setting data indicative of the three kinds of sound information pieces. Upon receipt at the processor 240 of the setting data, the processor 240 supplies to the audio signal generator 245 a sound information instruction instructing the audio signal generator 245 to play the three kinds of sound information pieces. Hereinbelow, it is assumed that first to third sound information pieces are used as the three kinds of sound information pieces. Each of the first audio signal generator 410, the second audio signal generator 420, and the third audio signal generator 430, upon receipt of a sound information instruction, operates as follows. The first audio signal generator 410 acquires the first sound information from the storage unit M, and generates digital stereo audio signals in a 2-channel format corresponding to the first sound information. The second audio signal generator 420 acquires the second sound information from the storage unit M, and generates digital stereo audio signals in a 2-channel format corresponding to the second sound information. The third audio signal generator 430 acquires the third sound information from the storage unit M, and generates digital stereo audio signals in a 2-channel format corresponding to the third sound information. It is of note that the audio signal generator 245 may consist of only the first audio signal generator 410. In this case, the setting data indicates a single kind of sound information.

The mixer 451 mixes (adds up) the left (L) audio signals output from respective ones of the first audio signal generator 410, the second audio signal generator 420, and the third audio signal generator 430, to generate an audio signal SD(L). Likewise, the mixer 452 mixes the right (R) audio signals output from respective ones of the first audio signal generator 410, the second audio signal generator 420, and the third audio signal generator 430, to generate an audio signal SD(R).

The effect imparter 250 shown in FIG. 2 generates an effect-imparted audio signal V by imparting an acoustic effect to the audio signal SD. The effect imparter 250 includes an effects unit. The acoustic effect includes an effect whereby frequency characteristics of an audio signal are changed over time, and also includes an effect whereby a level of distortion of an audio signal is changed over time. That is, the effect imparter 250 imparts to the audio signal SD an acoustic effect that changes over time, so as to generate the effect-imparted audio signal V with the effect imparted thereto. The changes in the acoustic effect are cyclical, and the effect imparter 250 is instructed by the processor 240 to impart the changes.

The effect imparter 250 in the present example is provided with a time varying filter F capable of changing at least frequency characteristics of an audio signal. As an example of the time varying filter F, a low-pass filter that passes frequency components of a low frequency range will be described. However, the time varying filter F may be a high-pass filter that passes frequency components of a high frequency range, or a band-pass filter that passes frequency components in a prescribed band.

FIG. 4 shows examples of time changes in a cut off frequency of a low-pass filter. As shown in FIG. 4, the cutoff frequency is a frequency f1 at a time point t0, and increases to a frequency f2 at a time point t1, and to a frequency f3 at a time point t2. Thereafter, the cutoff frequency decreases to a frequency f2 at a time point t3, and further down to a frequency f1 at a time point t4. We assume that the audio signal SD includes waveform data of a sound of ocean waves, the waveform data consisting of frequency components within a range from the frequency f1 to the frequency f2, and also assume that the audio signal includes waveform data of a bell sound, the waveform data consisting of frequency components within a range from the frequency f2 to the frequency f3. In this case, in a period from the time point t0 to the time point t1 and a period from the time point t3 to the time point t4, the sound of ocean waves is output but the bell sound is muted, and in a period from the time point t1 to the time point t3, the bell sound and the sound of ocean waves are output. Thus, in a case in which at least a portion of frequency components of the audio signal SD overlaps with a frequency range in which the cutoff frequency of the low-pass filter varies, sounds of the frequency components in the overlapping portion can either be played (output) or muted. Thus, even when using a single audio signal (sound content) consisting of waveform data of a sound of ocean waves and waveform data of a bell sound, as in the present example, variations can be created in an effect-imparted audio signal generated from the single audio signal.

The effect imparter 250 controls a timing at which to start changing a frequency characteristic, in accordance with a trigger signal supplied from the processor 240. The audio signal SD(L) to which there has been imparted an acoustic effect by the effect imparter 250, i.e., the effect-imparted audio signal V(L) is converted to an analog signal by the D/A converter 261, and the analog audio signal V(L) is supplied to the speaker 51. The audio signal SD(R) to which there has been imparted acoustic effect by the effect imparter 250, i.e., the effect-imparted audio signal V(R), is converted to an analog signal by the D/A converter 262, and the analog audio signal V(R) is supplied to the speaker 52.

The processor 240 activates the trigger signal at a switching cycle BRs that is in accordance with the breathing cycle BRm of the human subject E. FIG. 5 shows a relationship among a waveform of the audio signal SD, time changes in the cutoff frequency of the low-pass filter, and the trigger signals. Here, the switching cycle BRs that is in accordance with the breathing cycle BRm need not necessarily coincide with the detected breathing cycle BRm; it is sufficient if a particular relationship exists between the switching cycle BRs and the detected breathing cycle BRm. For example, a value obtained by multiplying an average value of breathing cycles BRm within a prescribed period by K (K is a freely-selected value satisfying 1≤K≤1.1) may be used as the switching cycle BRs. In the present example, the processor 240 sets as the switching cycle BRs a value obtained by multiplying the average value of the breathing cycles BRm by 1.05. In this case, given that an average breathing cycle BRm of the human subject E is 5 seconds, the switching cycle BRs is 5.25 seconds. The breathing cycle BRm tends to become longer as a person becomes relaxed. Accordingly, by employing a switching cycle BRs that is somewhat longer than the measured breathing cycle BRm, the human subject E can be guided to fall sleep.

In the example shown in FIG. 5, the cutoff frequency of the low-pass filter is the smallest at a timing at which each trigger signal is activated. Thus, the audio signal SD is attenuated to the greatest extent at a timing at which the trigger signal is activated. Accordingly, a volume of a sound that is audible to the human subject E and corresponds to the effect-imparted audio signal V becomes the lowest at a timing at which the trigger signal is activated. As a result, the human subject E is able to perceive his/her own breathing cycle BRm from a change in sound volume.

In the present example, the cutoff frequency changes based on the breathing cycle BRm of the human subject E; however, the cutoff frequency may change based on a switching cycle HRs that is in accordance with the heartbeat cycle HRm of the human subject E. Here, the switching cycle HRs that is in accordance with the heartbeat cycle HRm need not necessarily coincide with the detected heartbeat cycle HRm; it is sufficient if a particular relationship exists between the switching cycle HRs and the detected heartbeat cycle HRm. For example, a value obtained by multiplying an average value of heartbeat cycles HRm within a prescribed period by L (L is a freely-selected value satisfying 1≤L≤1.1) may be used as the switching cycle HRs. As an example, a value obtained by multiplying the average value of the heartbeat cycles HRm by 1.02 may be used as the switching cycle HRs. In this case, given that an average heartbeat cycle HRm of the human subject E is 1 second, the switching cycle is 1.02 seconds. The heartbeat cycle HRm tends to become longer as a person becomes relaxed. Accordingly, by employing a switching cycle HRs that is longer than the actual heartbeat cycle HRm, the human subject E can be relaxed towards sleep onset.

The effect imparter 250 imparts to the audio signal SD, in the manner described above, a frequency characteristic that changes at a cycle that is in accordance with either the breathing cycle BRm or the heartbeat cycle HRm, so as to generate the effect-imparted audio signal V. Accordingly, a variety of sounds can be output (played). In the preceding description, a low-pass filter is used as an example of the time varying filter F that varies the cutoff frequency over time; however, as the time varying filter F, there may be used a high-pass filter, or a band-pass filter.

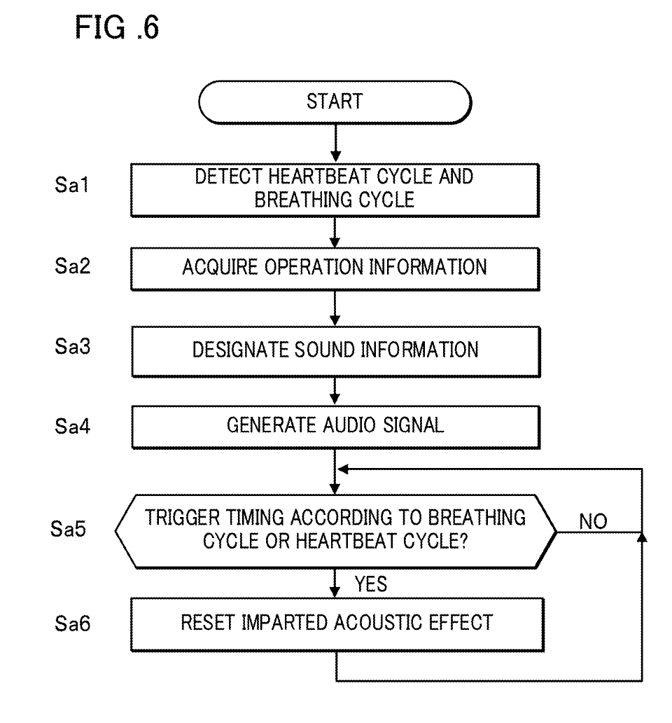

Next, an operation of the audio signal processing device 20 will be explained. FIG. 6 is a flowchart showing a flow of an operation of the audio signal processing device 20. First, the processor 240 detects the heartbeat cycle HRm and the breathing cycle BRm of the human subject E, based on the detected signal indicative of the biological information of the human subject E acquired by the acquirer 210 (Sa1). The frequency band including breathing components superimposed on the detected signal is within a range of from around 0.1 Hz to 0.25 Hz, while the frequency band including heartbeat components superimposed on the detected signal is within a range of from around 0.9 Hz to 1.2 Hz. The processor 240 extracts signal components in a frequency band corresponding to the breathing components, from the detected signal, and detects the breathing cycle BRm of the human subject E based on the extracted components. The processor 240 also extracts signal components in a frequency band corresponding to the heartbeat components, from the detected signal, and detects the heartbeat cycle HRm of the human subject E based on the extracted components. The processor 240 constantly detects the heartbeat cycle HRm and the breathing cycle BRm of the human subject E while the processes described below are being carried out.

The setter 220, upon acquiring the operation information from the input device 225 (Sa2), supplies to the processor 240 the setting data indicative of the sound information to be read from the storage unit M. The processor 240 supplies to the audio signal generator 245 a sound information instruction corresponding to the setting data, to designate sound information (Sa3). Thereafter, the audio signal generator 245 reads sound information according to the sound information instruction, and starts generating the audio signal SD using that sound information (Sa4).

Next, the processor 240 determines whether it is a trigger timing for the switching cycle BRs that is in accordance with the breathing cycle BRm of the human subject E or the switching cycle HRs that is in accordance with the heartbeat cycle HRm of the human subject E (Sa5). It may be determined in advance whether to employ as the trigger timing the switching cycle BRs that is in accordance with the breathing cycle BRm, or the switching cycle HRs that is in accordance with the heartbeat cycle HRm. Alternatively, the determination may be made based on the sound information designated in step Sa3.

When a determination condition of step Sa5 is not satisfied, the processor 240 repeats step Sa5. When the determination condition of step Sa5 is satisfied, the processor 240 activates the trigger signal. Accordingly, the effect imparter 250 resets the time changes of the acoustic effect to be imparted to the audio signal SD, and starts imparting the acoustic effect that has been set in advance to the audio signal SD (Sa6). Specifically, the frequency characteristic of the time varying filter F changes in accordance with a change in the cutoff frequency from the time point t0 onwards, as illustrated in FIG. 4.

As described in the foregoing, according to the present embodiment, time changes of an acoustic effect can be linked to a biorhythm, such as the breathing cycle BRm and the heartbeat cycle HRm, as a result of which variations in sound can be increased. That is, by changing an acoustic effect imparted to the audio signal and, in addition, modifying the time changes of the acoustic effect, an effect-imparted audio signal rich in variation can be created with simplicity from a single piece of sound information (sound content). Consequently, in the present embodiment, the human subject does not become bored by the output sound (sound that is played), and thus it is possible to guide the human subject to sleep.

### Second Embodiment

In the first embodiment described above, the effect imparter 250 imparts to the audio signal an acoustic effect that changes over time at a cycle that is in accordance with a biological cycle, such as the breathing cycle BRm and the heartbeat cycle HRm. In contrast, an audio signal processing device 20 according to the second embodiment estimates a sleep stage (sleep state) based on biological information of the human subject E, and in accordance with the estimated sleep stage, the effect imparter 250 additionally imparts a vibrato effect to the audio signal.

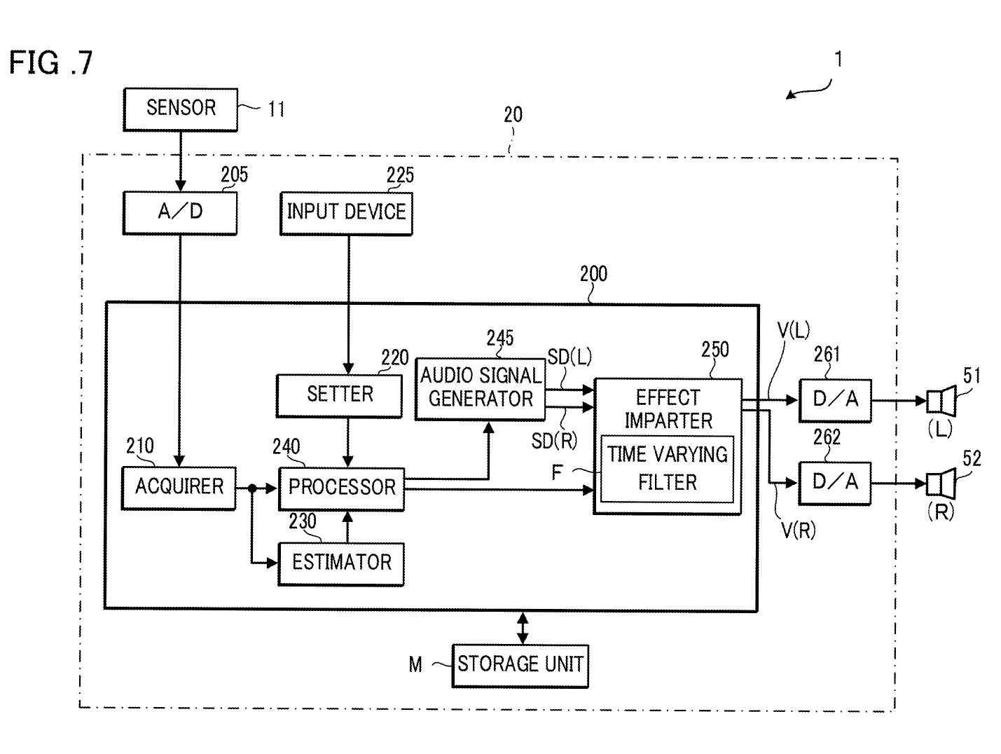

FIG. 7 is a block diagram showing an example configuration of a system 1 according to the second embodiment. The audio signal processing device 20 according to the second embodiment shown in FIG. 7 is configured in substantially the same way as the audio signal processing device 20 of the first embodiment, with the exception that the audio signal processing device 20 according to the second embodiment includes an estimator 230, and furthermore the processor 240 adjusts an acoustic effect based on an estimation result of the estimator 230 and a heartbeat cycle HRm, and, the effect imparter 250 imparts to the audio signal a vibrato effect as an acoustic effect in addition to changing a frequency characteristic of the audio signal. The term “vibrato effect”, in a narrow sense, is used to refer to an acoustic effect of modulating a frequency of an original sound at a vibrato cycle; however, in the present description, the term is used in a broader sense to refer to a concept inclusive of an effect that is referred to as “tremolo”, i.e. modulating amplitude of an original sound. That is, the vibrato effect in the present application also includes an acoustic effect of modulating amplitude of an original sound at a vibrato cycle (i.e., tremolo cycle).

In the present embodiment, the estimator 230 estimates from a detected signal of the sensor 11 a physical and mental state (sleep stages) of the human subject E in three stages, for example, over a period extending from a time point at which the human subject E enters a calm state and then falls asleep, until a time point at which the human subject E awakens. Generally speaking, as the state of a human changes from a calm state to deep sleep, the breathing cycle BRm and the heartbeat cycle HRm of the human tend to become longer, and fluctuations in the breathing cycle BRm and fluctuations in the heartbeat cycle HRm tend to become smaller. In addition, body motion becomes smaller as sleep deepens. With the foregoing taken into consideration, the estimator 230, based on the detected signals (biological information) from the sensor 11, obtains a value from changes in the breathing cycle BRm, changes in the heartbeat cycle HRm, and the number of body motion per unit time, and compares the obtained value with a plurality of thresholds to thereby estimate a sleep stage to be one of different sleep stages, namely, a first stage, a second stage, and a third stage.

In a state in which a human is active, almost all brainwaves are β waves. Then, when he/she becomes relaxed, α brain waves start to appear.

The frequency of α waves is 8 Hz to 14 Hz. For example, when a human goes to bed and closes his/her eyes, α waves start to appear. Then, as a human becomes more relaxed, α waves gradually increase. The period from when a human becomes relaxed to when α waves start to increase corresponds to the first stage, approximately. That is, the first stage is a stage before α waves become dominant.

Further, as a human descends into sleep, a proportion of α waves increases. Over time, however, α waves begin to decrease and θ waves start to appear. θ waves are said to appear when a human is in a state of meditation or is dozing. Approximately, a state up to this point corresponds to the second stage. That is, the second stage is a stage before θ waves become dominant. The frequency of θ waves is 4 Hz to 8 Hz.

θ waves subsequently become dominant, and a human substantially falls asleep. As sleep proceeds further, δ waves start to appear. δ waves are said to appear when a human is in a state of deep sleep. Approximately, a state up to this point corresponds to the third stage. That is, the third stage is a stage before δ waves become dominant. The frequency of δ waves is 0.5 Hz to 4 Hz.

The processor 240 decides a vibrato cycle in accordance with a sleep state estimated by the estimator 230. Then, the processor 240 causes the effect imparter 250 to impart to the audio signal a vibrato effect using the vibrato cycle.

In a case in which the estimation result of the estimator 230 is the first stage, the processor 240 supplies to the effect imparter 250 a first instruction that the vibrato cycle is to be set to a cycle corresponding to 8 Hz to 14 Hz, which is the frequency of α waves. The effect imparter 250, upon receipt of the first instruction, sets the vibrato cycle to a cycle corresponding to 8 Hz to 14 Hz, which is the frequency of α waves, in accordance with the first instruction. As described above, the first stage is a stage before α waves become dominant. Thus, by setting the vibrato cycle to a cycle corresponding to 8 Hz to 14 Hz, which is the frequency of α waves, a frequency fluctuation that corresponds to the frequency of α waves can be imparted to a sound that will be heard by the human subject E. Accordingly, the human subject E can be caused to further relax, and more readily be guided to a physical and mental state that is conducive to onset of sleep.

In a case in which the estimation result of the estimator 230 is the second stage, the processor 240 supplies to the effect imparter 250 a second instruction that the vibrato cycle is to be set to a cycle corresponding 4 Hz to 8 Hz, which is the frequency of θ waves. The effect imparter 250, upon receipt of the second instruction, sets the vibrato cycle to a cycle corresponding to 4 Hz to 8 Hz, which is the frequency of θ waves, in accordance with the second instruction. As described above, the second stage is a stage before θ waves become dominant. Thus, by setting the vibrato cycle to a cycle corresponding to 4 Hz to 8 Hz, which is the frequency of θ waves, a frequency fluctuation that corresponds to the frequency of θ waves can be imparted to a sound that will be heard by the human subject E. Accordingly, the human subject E can be caused to further relax, and more readily be guided to a physical and mental state that is conducive to onset of sleep.

In a case in which the estimation result of the estimator 230 is the third stage, the processor 240 supplies to the effect imparter 250 a third instruction indicating that the vibrato cycle is to be set to a cycle corresponding to 0.5 Hz to 4 Hz, which is the frequency of δ waves. The effect imparter 250, upon receipt of the third instruction, sets the vibrato cycle to a cycle corresponding to 0.5 Hz to 4 Hz, which is the frequency of δ waves, in accordance with the third instruction. As described above, the third stage is a stage before δ waves become dominant. Thus, by setting the vibrato cycle to a cycle corresponding to 0.5 Hz to 4 Hz, which is the frequency of δ waves, a frequency fluctuation that corresponds to the frequency of δ waves can be imparted to a sound that will be heard by the human subject E. Accordingly, the human subject E can be guided into deep sleep.

As described above, the processor 240 decides a cycle of vibrato to be imparted by the effect imparter 250 to the audio signal, in accordance with a sleep stage estimated by the estimator 230. The vibrato cycle is decided based on brainwaves (α, θ, and δ waves), and is not linked in particular to either a breathing cycle BRm or a heartbeat cycle HRm. As described below, however, the vibrato cycle may be changed so as to be linked to either the breathing cycle BRm or the heartbeat cycle HRm.

First, in a case in which the estimation result of the estimator 230 is the first stage, the processor 240 sets the vibrato cycle to have a frequency of α waves as follows. The frequency of α waves is 8 Hz to 14 Hz. This frequency (8 Hz to 14 Hz) corresponds to a time interval between one beat and a next beat at a musical tempo of 480 to 840 BPM (hereinafter, “first interval”). Thus, the first interval corresponds to the cycle of α waves. The heartbeat cycle HRm during a calm state, when converted to a musical tempo, corresponds to a musical tempo of about 60 to 75 BPM. Thus, the heartbeat cycle HRm during a calm state (corresponding to a musical tempo of about 60 to 75 BPM) is about eight times the cycle of α waves (corresponding to a musical tempo of 480 to 840 BPM). In other words, a musical tempo of 60 to 105 BPM has a cycle that is one-eighth of the cycle of a musical tempo of 480 to 840 BPM that corresponds to α waves. Then, given that a beat in a musical tempo of 60 to 105 BPM corresponds to a quarter note (i.e., given that a time interval between one beat and a next beat at a musical tempo of 60 to 105 BPM is equivalent to a period represented by a quarter note), the first interval (corresponding to a cycle of α waves) is an interval that is equivalent to a period represented by a thirty second note. Thus, given that a tempo of a sound that will be heard by the human subject E is set as a musical tempo of 60 to 75 BPM (the heartbeat cycle HRm during a calm state), and a beat in this musical tempo of 60 to 75 BPM corresponds to a quarter note, the following effect can be achieved. Namely, a frequency fluctuation that is linked to the heartbeat cycle HRm and that corresponds to α waves can be realized by adding, to the sound that will be heard by the human subject E, vibrato having a cycle corresponding to an interval that is equivalent to a period represented by a thirty second note. Given that the vibrato cycle is VIs, the vibrato cycle VIs in a case in which the estimation result of the estimator 230 is the first stage is given by equation 1 below.

VIs=HRm/N1 equation 1

Here, N1 is a natural number of 6 to 14 (inclusively). In a case that N1=8, vibrato corresponding to a thirty second-note interval is realized. As described above, by setting the vibrato cycle VIs as a value obtained by dividing the heartbeat cycle HRm by a natural number N1 in an appropriate range, there can be obtained a vibrato cycle VIs that is linked to the heartbeat cycle HRm (linked to one over natural number of the heartbeat cycle) and falls within a range of the frequency 8 Hz to 14 Hz of α waves.

In a case in which the estimation result of the estimator 230 is the second stage, the processor 240 sets the vibrato cycle to have a frequency of θ waves as follows. The frequency of θ waves is 4 Hz to 8 Hz. This frequency (4 Hz to 8 Hz) corresponds to an interval between one beat and a next beat in a musical tempo of 240 to 480 BPM (hereinafter, “second interval”). Thus, the second interval corresponds to the cycle of θ waves. As described above, the heartbeat cycle HRm during a calm state, when converted to a musical tempo, is about 60 to 75 BPM. Thus, the heartbeat cycle HRm during a calm state (corresponding to a musical tempo of about 60 to 75 BPM) is about four times the cycle of θ waves (a musical tempo of 240 to 480 BPM). Here, given that a beat in a musical tempo of 60 to 120 BPM having a cycle that is one-fourth of the cycle of a musical tempo of 240 to 480 BPM corresponding to θ waves is equivalent to a quarter note (i.e., given that a time interval between one beat and a next beat in a musical tempo of 60 to 120 BPM is equivalent to a period represented by a quarter note), the second interval (corresponding to the cycle of θ waves) is an interval that is equivalent to a period represented by a sixteenth note. Thus, given that a tempo of a sound that will be heard by the human subject E is set to be a musical tempo of 60 to 75 BPM (the heartbeat cycle HRm during a calm state), and a beat in this musical tempo of 60 to 75 BPM corresponds to a quarter note, the following effect can be achieved. Namely, a frequency fluctuation that is linked to the heartbeat cycle HRm and that corresponds to θ waves is realized by adding, to the sound that will be heard by the human subject E, vibrato having a cycle corresponding to an interval that is equivalent to a period represented by a sixteenth note.

The vibrato cycle VIs in a case in which the estimation result of the estimator 230 is the second stage is given by equation 2 below.

VIs=HRm/N2 equation 2

Here, N2 is a natural number of 2 to 8 (inclusively).

In a case that N2=4, vibrato corresponding to a sixteenth-note interval is realized. As described above, by setting the vibrato cycle VIs as a value obtained by dividing the heartbeat cycle HRm by a natural number N2 in an appropriate range, a vibrato cycle VIs that is linked to the heartbeat cycle HRm (linked to one over natural number of the heartbeat cycle) and falls within a range of the frequency 4 Hz to 8 Hz of θ waves can be obtained. The ranges of N1 and N2 can be modified, as appropriate.

In a case in which the estimation result of the estimator 230 is the third stage, the processor 240 sets the vibrato cycle to have a frequency of δ waves as follows. The frequency of δ waves is 0.5 Hz to 4 Hz. This frequency (0.5 Hz to 4 Hz) corresponds to an interval between one beat and a next beat in a musical tempo of 30 to 240 BPM (hereinafter, “third interval”). Thus, the third interval corresponds to the cycle of δ waves. Here, a musical tempo of 60 to 75 BPM corresponding to the heartbeat cycle HRm during a calm state is included in a musical tempo of 30 to 240 BPM corresponding to δ waves, and therefore, given that a beat in a musical tempo of about 60 to 75 BPM corresponding to the heartbeat cycle HRm during a calm state corresponds to a quarter note, the third interval (corresponding to the cycle of δ waves) is an interval that is equivalent to a period represented by a quarter note. That is, the third interval (corresponding to the cycle of δ waves) corresponds to the heartbeat cycle HRm. Thus, the processor 240 need only use the heartbeat cycle HRm as it is, to attain the vibrato cycle VIs. In a case in which an acoustic effect is imparted at a cycle that is in accordance with the heartbeat cycle HRm, as described in the first embodiment above, an audio signal already has frequency fluctuation corresponding to δ waves. Thus, the processor 240 need not issue an instruction to further impart a vibrato effect, to the effect imparter 250. On the other hand, in a case in which the effect imparter 250 is imparting the effect according to the effect impartation of the first embodiment at a cycle that is in accordance with the breathing cycle BRm, then in addition to the effect impartation set out in the first embodiment, the processor 240 may supply to the effect imparter 250 the third instruction indicating that the vibrato effect having the vibrato cycle VIs corresponding to the heartbeat cycle HRm is to be imparted to the audio signal SD. In this case, the effect imparter 250, upon receipt of the third instruction, sets the vibrato cycle to a cycle that is linked to the heartbeat cycle HRm (linked to one over natural number of the heartbeat cycle) and that falls within a range of the frequency 0.5 Hz to 4 Hz of δ waves, in accordance with the third instruction.

As described above, the processor 240 can set the vibrato cycle VIs to be a cycle that is in accordance with the heartbeat cycle HRm and that is a cycle of brainwaves predicted to appear when a sleep state becomes deeper than a current sleep state.

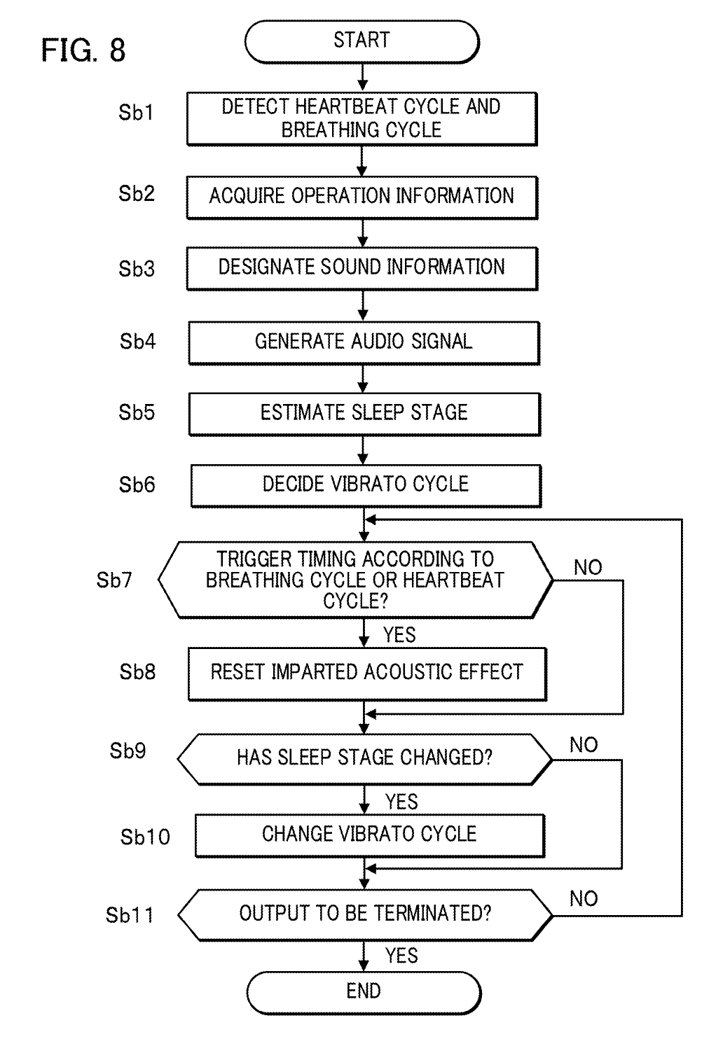

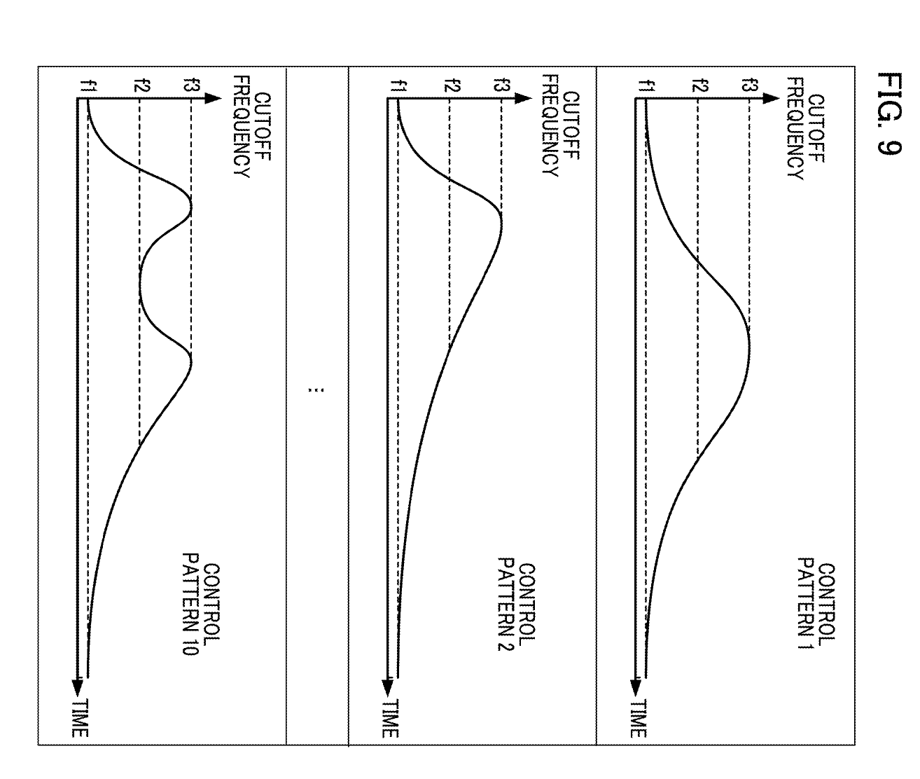

Next, an operation of the audio signal processing device 20 of the second embodiment will be explained. FIG. 8 is a flowchart showing a flow of an operation of the audio signal processing device 20. Processes in steps Sb1 to Sb4 are substantially the same as those in steps Sa1 to Sa4 described with reference to FIG. 6 and pertaining to the operation of the audio signal processing device 20 of the first embodiment, and thus description thereof will be omitted.

In step Sb5, the estimator 230 estimates a sleep stage of the human subject E based on the biological information. Next, the processor 240 decides the vibrato cycle VIs in accordance with the estimation result of the estimator 230 (Sb6). Next, the processor 240 supplies to the effect imparter 250 an instruction indicating that a vibrato effect having the vibrato cycle VIs is to be imparted to audio signals SD. The effect imparter 250, upon receipt of the instruction from the processor 240, generates effect-imparted audio signals V in accordance with the instruction. Next, the effect imparter 250 outputs the effect-imparted audio signals V to D/A converters 261 and 262. The effect-imparted audio signals V are converted by the D/A converters 261 and 262 to analog signals, and sounds corresponding to the analog effect-imparted audio signals V are output from the speakers 51 and 52.

Next, the processor 240 either executes step Sb7 or executes steps Sb7 and Sb8. Processes in steps Sb7 and Sb8 are substantially the same as those in steps Sa5 to Sa6 described with reference to FIG. 6 and pertaining to the operation of the audio signal processing device 20 of the first embodiment, and thus description thereof will be omitted. Next, the processor 240 determines whether the sleep stage has changed (Sb9). When the sleep stage has changed, the processor 240 supplies to the effect imparter 250 an instruction indicating that a vibrato effect having a vibrato cycle VIs that corresponds to the sleep stage after the change is to be imparted to the audio signal SD, and thereby changes the currently-set vibrato cycle to a vibrato cycle that corresponds to the sleep stage after the change (Sb10). Thereafter, the processor 240 determines whether output of sound is to be terminated (Sb11), and when a determination condition of step Sb11 is not satisfied, the processor 240 returns the process to step Sb7. When the determination condition of step Sb11 is satisfied, the processor 240 terminates the process.

As described above, according to the present embodiment, a sleep stage is estimated, and a vibrato effect is imparted to an audio signal SD at a vibrato cycle that corresponds to the estimated sleep stage. Since the vibrato cycle is a cycle in accordance with a frequency of brainwaves that will be dominant in the subsequent sleep stage, it is possible to guide the human subject E to the subsequent sleep stage and in turn rapidly induce sleep in the human subject E. Moreover, by making the vibrato cycle VIs a cycle that is in accordance with the heartbeat cycle HRm (one over natural number of the heartbeat cycle HRm), a fluctuation of a frequency that is linked to a biological cycle derived from the human subject E can be imparted to the sound that corresponds to the effect-imparted audio signal V, and quality of sleep of the human subject E can be further enhanced.

### Modifications

The present invention is not limited to the embodiments described above, and various applications and modifications thereof, such as those described below, are possible. Furthermore, a freely selected one or a plurality of modifications of the applications and modifications described below may be combined, as appropriate.

### Modification 1

In the embodiments described above, a sheet-like sensor 11 is used to detect biological information of the human subject E. However, a sensor for detecting biological information of the human subject E is not limited to a sheet-like sensor 11, and a freely selected sensor may be used in so far as the sensor is capable of detecting biological information. For example, a brainwave sensor may be used as a sensor for detecting biological information of the human subject E. In this case, an electrode of the brainwave sensor is attached to, for example, the forehead of the human subject E to detect brainwaves (e.g., α waves, β waves, δ waves, and θ waves) of the human subject E, for example. Moreover, a pulse wave sensor may be used as a sensor for detecting biological information of the human subject E. In this case, the pulse wave sensor is attached to a wrist of the human subject E to detect pressure changes in the radial artery, i.e., pulse waves, for example. Since pulse waves are synchronous with heartbeats, detection of pulse waves means indirectly detecting heartbeats. Furthermore, an acceleration sensor may be used as a sensor for detecting biological information of the human subject E. In this case, the acceleration sensor may be provided between the head of the human subject E and a pillow to detect breathing, a heartbeat, and the like from body motion of the human subject E, for example.

In the embodiments described above, the breathing cycle BRm and the heartbeat cycle HRm are identified based on the biological information output from the sensor 11. However, the present invention is not limited thereto. At least one of the breathing cycle BRm and the heartbeat cycle HRm of the human subject may be identified, and to the audio signal SD there may be imparted an acoustic effect having a frequency characteristic that changes at a cycle that is in accordance with either of them (the identified breathing cycle BRm or the identified heartbeat cycle HRm).

### Modification 2

In the first embodiment described above, an acoustic effect that changes over time at a cycle that is in accordance with either the breathing cycle BRm or the heartbeat cycle HRm is imparted to the audio signal SD. The time changes of such acoustic effect are such as those shown in FIG. 4 for example, and are fixed. However, the present invention is not limited thereto. The processor 240 may randomly select a control pattern indicating time changes of an acoustic effect, from among a plurality of such control patterns. For example, as shown in FIG. 9, ten control patterns may be stored in the storage unit M in advance, and the processor 240 may randomly switch from one control pattern to another among the ten control patterns, at a cycle that is in accordance with either the breathing cycle BRm or the heartbeat cycle HRm. A concept of randomness includes so-called pseudo randomness, and the processor 240 may use pseudorandom signals generated by a maximal length sequence generator, to perform a variety of selections. By randomly switching control patterns as described above, variations in sounds to be output (played) can be increased. Thus, even when a number of sound information pieces stored in the storage unit M is small, sounds (playback sounds) that do not cause boredom in the human subject E can be played so as to be heard by the human subject E.

In the first embodiment described above, the effect imparter 250 imparts an acoustic effect that changes over time to the audio signal SD, at a cycle that is in accordance with either the breathing cycle BRm or the heartbeat cycle HRm. The present invention is not limited thereto, and the effect imparter 250 may impart an acoustic effect that changes over time to the audio signal SD, at a cycle that is in accordance with a biological cycle linked to biological activities of the human subject E.

### Modification 3

In the second embodiment described above, a fixed cycle or a cycle that is linked to the heartbeat cycle HRm is used as the vibrato cycle VIs. However, the present invention is not limited thereto. For example, from among biological cycles that are linked to biological activities of the human subject E and are obtained from biological information, the vibrato cycle VIs may be linked to one that is other than the heartbeat cycle HRm. For example, the vibrato cycle VIs may be linked to the breathing cycle BRm. In this case, a vibrato cycle VIs used in the first stage for inducing α waves is given by equation 3 below.

VIs=BRm/N3 equation 3

Here, N3 is a natural number of 30 to 70 (inclusively).

Equation 3 functions as a transformation for transforming a breathing cycle (BRm) during a calm state to a cycle (VIs in equation 3) of α waves.

A vibrato cycle VIs used in the second stage for inducing θ waves is given by equation 4 below.

VIs=BRm/N4 equation 4

Here, N4 is a natural number of 10 to 40 (inclusively).

Equation 4 functions as a transformation for transforming a breathing cycle (BRm) during a calm state to a cycle (VIs in equation 4) of θ waves.

A vibrato cycle VIs used in the third stage for inducing δ waves is given by equation 5 below.

VIs=BRm/N5 equation 5

Here, N5 is a natural number of 5 to 10 (inclusively).

Equation 5 functions as a transformation for transforming a breathing cycle (BRm) during a calm state to a cycle (VIs in equation 5) of δ waves. Here, by dividing the breathing cycle BRm by a corresponding one of N3, N4, and N5 in each of equations 3 to 5, an appropriate vibrato cycle VIs that is linked to the breathing cycle BRm (i.e., one over natural number of the breathing cycle BRm) is obtained. The range of each of N3, N4, and N5 may be changed as appropriate.

### Modification 4

The effect imparter 250 of the second embodiment described above imparts the vibrato effect in addition to the acoustic effect of the first embodiment, but the present invention is not limited thereto. For example, the effect imparter 250 of the second embodiment may impart the vibrato effect of the second embodiment without imparting the acoustic effect of the first embodiment. That is, the audio signal processing device may be an audio signal processing device including: an acquirer configured to acquire biological information of a human subject; an estimator configured to estimate a sleep state based on the biological information; a processor (controller) configured to decide a vibrato cycle in accordance with the sleep state estimated by the estimator; and an effect imparter configured to impart to an audio signal a vibrato effect corresponding to the vibrato cycle decided by the processor.

Moreover, the vibrato effect may be imparted to the audio signal SD by the audio signal generator 245 instead of being imparted to the audio signal SD by the effect imparter 250. In this case, when the audio signal generator 245 is constituted of a plurality of audio signal generators as shown in FIG. 3, at least one of those may impart a vibrato effect to the audio signal. Here, the audio signal generator 245 imparts the vibrato effect to the audio signal in accordance with an instruction from the processor 240.

The estimator 230 described above estimates a sleep state by dividing the sleep state into three stages. However, the present invention is not limited thereto. For example, the estimator 230 may estimate a sleep state by dividing the sleep state into two or more stages, or may estimate an index indicative of a degree of depth of sleep. In short, it is sufficient if the estimator 230 is capable of estimating a sleep state of the human subject E and the processor 240 is capable of modifying the acoustic effect (e.g., vibrato effect) that changes over time, in accordance with the estimated sleep state.

Additionally, an acoustic effect of changing a panning of a sound may be used as the acoustic effect of the first embodiment that changes over time. Specifically, a position of the panning of the sound may be switched as L→R→L→R . . . at switching cycles BRs or HRs. Moreover, pitch changes that change a pitch of a sound at a switching cycle BRs or HRs may be used as an acoustic effect that changes over time.

The following modes are derived from at least one of the embodiments and modifications described above.

The effect imparter 250 is provided with a time varying filter F, which is capable of changing a cutoff frequency for the audio signal SD, and changes the cut-off frequency at a cycle that is in accordance with either the breathing cycle BRm or the heartbeat cycle HRm.

In this mode, the cutoff frequency of the time varying filter F is changed over time at a cycle that is in accordance with a biological cycle, such as the breathing cycle BRm or the heartbeat cycle HRm, as a result of which a variety of sounds can be generated. In particular, in a case in which the time varying filter F is a low-pass filter or a high-pass filter, if a frequency range of a certain sound included in the audio signal SD corresponds to a part of a frequency range within which the cutoff frequency of the low-pass filter or the high-pass filter changes, then variations, such as the sound being played (output) and muted, can be created.

The processor 240 decides a vibrato cycle in accordance with the sleep state estimated by the estimator 230, and the effect imparter 250 imparts to the audio signal a vibrato effect that corresponds to the vibrato cycle decided by the processor 240.

In this mode, a vibrato effect with a vibrato cycle that corresponds to a sleep state can be imparted to the audio signal.

The processor 240 sets the vibrato cycle to a cycle of brainwaves that are predicted to appear in a case in which a sleep state becomes deeper than a current sleep state.

In this mode, a cycle of brainwaves that are predicted to appear in a case in which sleep becomes deeper is used as the vibrato cycle, whereby it is possible to guide the human subject E to fall asleep, and after the human subject E has fallen asleep, it is possible to guide the human subject E to a deeper sleep.

The processor 240 sets the vibrato cycle to one over natural number of either the heartbeat cycle or the breathing cycle.

In this mode, the vibrato cycle is set to one over natural number of either the heartbeat cycle or the breathing cycle of the human subject, and therefore, a fluctuation of a frequency that is linked to a biological cycle derived from the human subject E hearing the output sound (playback sound) can be imparted to the output sound, and a quality of sleep of the human subject E can be further enhanced.

The following configurations may be envisaged from the embodiments described above. That is, in one aspect, an audio signal processing device of the present invention includes: an acquirer configured to acquire biological information of a human subject; a processor configured to identify at least one of a breathing cycle and a heartbeat cycle of the human subject, based on the biological information; and an effect imparter configured to impart to an audio signal a frequency characteristic that changes at a cycle that is in accordance with either the breathing cycle or the heartbeat cycle.

In this aspect, time changes in the frequency characteristic of the audio signal are linked to a biorhythm, such as a breathing cycle and a heartbeat cycle. Consequently, it is possible to increase variations in the sound generated from the audio signal. The description “the time changes in the frequency characteristic of the audio signal are linked to a biorhythm, such as a breathing cycle and a heartbeat cycle” means that “the frequency characteristic of the audio signal changes in accordance with a biorhythm, such as a breathing cycle and a heartbeat cycle”. Moreover, a sound that is rich in variation can be created with simplicity from a single audio signal. As such, the human subject does not become bored by a sound corresponding to the audio signal that has been processed by the audio signal processing device, and the human subject can be guided toward sleep with relative ease. Thus, there can be avoided sleep disturbance in the human subject upon hearing a sound that actually is intended to enhance sleep in the human subject. Here, the cycle that is in accordance with either the breathing cycle or the heartbeat cycle need not be the actual breathing cycle or heartbeat cycle itself. It is sufficient if a particular relationship exists between this cycle and the breathing cycle or the heartbeat cycle.

In another aspect, an audio signal processing device according to the present invention includes: an acquirer configured to acquire biological information of a human subject; an estimator configured to estimate a sleep state based on the biological information; a processor configured to decide a vibrato cycle in accordance with the sleep state estimated by the estimator; and an effect imparter configured to impart to an audio signal a vibrato effect corresponding to the vibrato cycle decided by the processor.

In this aspect, the vibrato effect can be changed such that, for example, a sleep state of the human subject becomes deeper than a current state.

The present invention has been described with reference to embodiments, but the present invention is not limited to the above embodiments. Various changes comprehensible to a person skilled in the art can be made to the configurations and details of the present invention, so long as they remain within the scope of the present invention. The present application claims priority based on Japan Patent Application No. 2015-130156 filed on Jun. 29, 2015, the entire contents of which are incorporated herein.

### DESCRIPTION OF REFERENCE SIGNS

1: system
11: sensor
20: audio signal processing device
245: audio signal generator
51, 52: speakers
210: acquirer
220: setter
230: estimator
240: processor
250: effect imparter
M: storage unit
F: time varying filter

1. An audio signal processing device, comprising:
an acquirer configured to acquire biological information of a human subject;
a processor configured to identify at least one of a breathing cycle and a heartbeat cycle of the human subject, based on the biological information; and
an effect imparter configured to impart to an audio signal a frequency characteristic that changes at a cycle that is in accordance with either the breathing cycle or the heartbeat cycle.

2. The audio signal processing device according to claim 1, wherein
the effect imparter
includes a time varying filter for changing a cut-off frequency for the audio signal, and
changes the cut-off frequency at a cycle that is in accordance with either the breathing cycle or the heartbeat cycle.

3. The audio signal processing device according to claim 1, further comprising:
an estimator configured to estimate a sleep state based on the biological information, wherein
the processor decides a vibrato cycle in accordance with the sleep state estimated by the estimator, and
the effect imparter additionally imparts to the audio signal a vibrato effect corresponding to the vibrato cycle decided by the processor.

4. The audio signal processing device according to claim 3, wherein
the processor sets the vibrato cycle to a cycle of a brainwave that is predicted to appear in a case in which a sleep state becomes deeper than a current sleep state.

5. The audio signal processing device according to claim 3, wherein
the processor sets the vibrato cycle to one over natural number of either the heartbeat cycle or the breathing cycle.

6. The audio signal processing device according to claim 4, wherein
the processor sets the vibrato cycle to one over natural number of either the heartbeat cycle or the breathing cycle.

7. An audio signal processing device, comprising:
an acquirer configured to acquire biological information of a human subject;
an estimator configured to estimate a sleep state based on the biological information;
a processor configured to decide a vibrato cycle in accordance with the sleep state estimated by the estimator; and
an effect imparter configured to impart to an audio signal a vibrato effect corresponding to the vibrato cycle decided by the processor.

8. An audio signal processing method, comprising:
acquiring biological information of a human subject;
identifying at least one of a breathing cycle and a heartbeat cycle of the human subject, based on the biological information; and
imparting to an audio signal a frequency characteristic that changes at a cycle that is in accordance with either the breathing cycle or the heartbeat cycle.

9. An audio signal processing method, comprising:
acquiring biological information of a human subject;
estimating a sleep state based on the biological information;
deciding a vibrato cycle in accordance with the sleep state; and
imparting to an audio signal a vibrato effect corresponding to the vibrato cycle.

10. A non-transitory computer-readable storage medium having stored therein a program for causing a computer to execute:
an acquisition procedure of acquiring biological information of a human subject;
a processing procedure of identifying at least one of a breathing cycle and a heartbeat cycle of the human subject, based on the biological information; and
an effect impartation procedure of imparting to an audio signal a frequency characteristic that changes at a cycle that is in accordance with either the breathing cycle or the heartbeat cycle.

11. A non-transitory computer-readable storage medium having stored therein a program for causing a computer to execute:
an acquisition procedure of acquiring biological information of a human subject;
an estimation procedure of estimating a sleep state based on the biological information;
a processing procedure of deciding a vibrato cycle in accordance with the sleep state; and
an impartation procedure of imparting to an audio signal a vibrato effect corresponding to the vibrato cycle.
